(19) United States
(12) Patent Application Publication
(10) Pub. No.: US 2008/0281130 A1
(43) Pub. Date: Nov. 13, 2008

(54) PROCESS FOR PRODUCING BISPHENOL A WITH AN EXTENDED SERVICE LIFE IN THE CRYSTALLISATION

(75) Inventors: Ulrich Blaschke, Krefeld (DE); Raymond Audenaert, Hamme (BE); Jeroen Geers, Hoogerheide (NL); Philip De Kock, Kapellen (BE); Paul Salembier, Tielrode (BE); Dirk Petein, Kruibeke (BE)

Correspondence Address: CONNOLLY BOVE LODGE & HUTZ, LLP, P O BOX 2207, WILMINGTON, DE, 19899, US

(73) Assignee: Bayer MaterialScience AG, Leverkusen (DE)

(21) Appl. No.: 12/115,655

(22) Filed: May 6, 2008

(30) Foreign Application Priority Data
May 10, 2007 (DE) 102007021935.2

(51) Int. Cl. C07C 37/68

(52) U.S. Cl. 568/724

(57) ABSTRACT

The invention describes a process for producing bisphenol with a content of less than 15 wt. % of phenol, wherein a suspension crystallisation of a product mixture containing bisphenol A, minor components and phenol is performed in a crystalliser, the product mixture being pumped through a heat exchanger. Due to deposition (fouling, i.e. crystallisation and deposition on the surface of the heat exchangers) in the heat exchanger, the pressure difference increases from 0.5 to 3 bar across the heat exchanger. The resulting reduced flow rate, which would lead to increased fouling in the crystalliser, is offset by continuously increasing the speed of the pump, the pump speed being regulated in such a way that the current consumption of the pump is maintained such that it fluctuates by a maximum of 5%.

### RELATED APPLICATIONS

This application claims benefit to German Patent Application No. 10 2007 021 935, filed May 10, 2007, which is incorporated herein by reference in its entirety for all useful purposes.

### BACKGROUND OF THE INVENTION

The invention describes a process for producing bisphenol with a content of less than 15 wt. % of phenol, wherein a suspension crystallisation of a product mixture containing bisphenol A, minor components and phenol is performed in a crystalliser, the product mixture being pumped through a heat exchanger. Due to deposition (fouling, i.e. crystallisation and deposition on the surface of the heat exchangers) in the heat exchanger, the pressure difference increases from 1 to 3 bar across the heat exchanger. The resulting reduced flow rate, which would lead to increased fouling in the crystalliser, is offset by continuously increasing the speed of the pump, the pump speed being regulated in such a way that the current consumption is maintained such that it fluctuates by a maximum of ±5%.

As condensation products of phenols and carbonyl compounds, bisphenols are starting products or intermediates for the production of many commercial products. Of particular industrial importance is the condensation product from the reaction between phenol and acetone, 2,2-bis(4-hydroxyphenyl)propane (bisphenol A, BPA). BPA is used as a starting product for the production of various polymeric materials, such as for example polyarylates, polyetherimides, polysulfones and modified phenol-formaldehyde resins. Preferred areas of application are in the production of epoxy resins and polycarbonates.

Industrially relevant production methods for BPA are known and are based on the acid-catalysed reaction of phenol with acetone, and are described for example in U.S. Pat. No. 2,775,620 and in EP-A 0 342 758.

In the acid-catalysed reaction of phenol with acetone a phenol to acetone ratio of greater than 5:1 is preferably established in the reaction. The reaction is conventionally performed continuously and generally at temperatures of 45 to 110° C. Both homogeneous and heterogeneous Brønsted or Lewis acids can be used as acid catalysts, such as for example strong mineral acids such as hydrochloric or sulfuric acid. Gel-like or macroporous sulfonated crosslinked polystyrene resins (acid ion exchangers) are preferably used which contain divinylbenzene as the crosslinking agent. In addition to the catalyst, a thiol is generally used as a co-catalyst.

In the reaction of phenol with acetone in the presence of acid catalysts, a product mixture is formed which primarily contains, in addition to unreacted phenol and possibly acetone, BPA and water. In addition, typical by-products of the condensation reaction occur, such as for example 2-(4-hydroxyphenyl)-2-(2-hydroxyphenyl) propane (o,p-BPA), substituted indanes, hydroxyphenyl indanols, hydroxyphenyl chromanes, spiro bis-indanes, substituted indenols, substituted xanthenes and more highly condensed compounds having three or more phenyl rings in the molecular skeleton. In addition, further minor components such as anisol, mesityl oxide, mesitylene and diacetone alcohol can form as a result of natural condensation of the acetone and reaction with impurities in the raw materials.

The cited secondary products such as water, but also the unreacted feed materials such as phenol and acetone, have a detrimental effect on the suitability of BPA to produce polymers and must be separated off by means of suitable methods. In particular, high purity requirements are set for the raw material BPA for the production of polycarbonate.

One method of processing and purifying BPA involves separating BPA out of the product mixture in the form of an approximately equimolar crystalline adduct with phenol by cooling the reaction mixture and crystallising out the BPA-phenol adduct in a suspension crystallisation. The BPA-phenol adduct crystals are then separated from the liquid phase by means of suitable apparatus for solid/liquid separation such as rotary filters or centrifuges and sent for further purification.

In the suspension crystallisation, heat is extracted continuously or semi-continuously from the product mixture containing bisphenol A and phenol in one or more coolers to produce a supersaturation such that the BPA-phenol adduct crystals then crystallise out. The residence time necessary for breaking down the supersaturation and hence for crystallisation is provided in a crystalliser in addition to the coolers. The suspension from the crystalliser is generally circulated through the cooler(s) by pumps. A stream is released continuously or semi-continuously from the suspension for further processing, likewise fresh product mixture is added from the reaction, wherein water, acetone and other highly volatile components such as co-catalyst can be completely or partially removed from the product mixture in advance by distillation.

Processes for producing bisphenols with such a suspension crystallisation are described in DE-A 100 15 014 and in DE-A 195 10 063.

These publications also mention that in such a suspension crystallisation by means of circulating coolers, deposits consisting of bisphenol or BPA-phenol adduct crystals, for example, are deposited on the inner surface of the suspension-carrying tubes (known as fouling). The products adhering to the inner surface of the suspension-carrying tubes have to be removed at regular intervals by heating to temperatures above 80° C. (known as melt-off), since they reduce the transfer of heat between the cooler and the suspension and the flow through the tubes.

U.S. Pat. No. 5,856,589 describes the removal of such deposits by means of phenol with a water content of 5 to 40 wt. %.

U.S. Pat. No. 6,203,612 describes a further, complicated process for removing the deposits, wherein part of the suspension contained in the crystalliser or cooler is first replaced by phenol and the diluted suspension is heated until the crystals are dissolved. The mixture is then quickly cooled and seed crystals are added.

WO-A 00/47542 describes the running of a suspension crystallisation in which a crystalliser contains two coolers, wherein in order to remove the deposits one cooler is taken out of operation and freed from deposits by increasing the temperature whilst the other cooler remains in operation and its temperature is lowered by at least 1° C.

These applications are all concerned with the removal of deposits. During this time bisphenol production has to be reduced or halted altogether.

### EMBODIMENTS OF THE INVENTION

An embodiment of the present invention is a process for producing bisphenol A comprising (a) reacting phenol and acetone in the presence of a sulfonic acid ion exchanger and a sulfur-containing co-catalyst to form a product mixture comprising bisphenol A-phenol adduct; (b) subjecting said product mixture comprising bisphenol A-phenol adduct obtained in a) to continuous suspension crystallisation in one or more crystallisers, wherein said one or more crystallisers comprises at least one heat exchanger for cooling and at least one pump which pumps the suspension contained in said one or more crystallisers through said at least one heat exchanger, to obtain crystals of bisphenol A-phenol adduct; (c) separating said crystals of bisphenol A-phenol adduct obtained in b) by solid/liquid separation; and (d) removing phenol from said crystals of bisphenol A-phenol adduct obtained in c) by distillation and/or desorption to obtain bisphenol A; wherein the current consumption of said at least one pump in b) is maintained such that the current consumption fluctuates by a maximum of ±5% and wherein the speed of said at least one pump is increased as the pressure loss across said at least one heat exchanger rises.

Another embodiment of the present invention is the above process, wherein the separated crystals of bisphenol A-phenol adduct obtained in c) are washed with phenol and/or recrystallised.

Another embodiment of the present invention is the above process, wherein said bisphenol A obtained in d) has a residual phenol content of <200 ppm.

Another embodiment of the present invention is the above process, wherein said bisphenol A obtained in d) has a residual phenol content of 15%.

Another embodiment of the present invention is the above process, wherein the speed of said at least one pump is increased by 10 to 50% from start-up of said at least one heat exchanger through to removal of the deposits in said at least one heat exchanger, relative to the initial speed.

Another embodiment of the present invention is the above process, wherein the volume throughput circulated through said at least one heat exchanger is maintained such that the current consumption fluctuates by a maximum of ±15%.

Another embodiment of the present invention is the above process, wherein the pressure difference across said at least one heat exchanger is measured and the speed of said at least one pump is increased as the pressure difference rises.

### DESCRIPTION OF THE INVENTION

The object of this invention was therefore to provide a process which allows the length of time between operations to remove the deposits to be increased. This object is achieved by a process for producing bisphenol A comprising the following steps:
a) Reaction of phenol and acetone in the presence of a sulfonic acid ion exchanger and a sulfur-containing co-catalyst to form a product mixture containing bisphenol A,
b) Continuous suspension crystallisation of a bisphenol A-phenol adduct from the product mixture obtained in step (a) in one or more crystallisers, each having at least one heat exchanger for cooling and at least one pump which pumps the suspension contained in the crystallisers through the heat exchanger(s),
c) Separation of the BPA-phenol adduct crystals obtained in this way by solid/liquid separation,
d) Removal of phenol by distillation and/or desorption from the BPA-phenol adduct obtained according to step (c),
characterised in that the current consumption of the pump(s) used to pump the product mixture in step b) through the heat exchangers is maintained such that the current consumption fluctuates by a maximum of +5% and hence as the pressure loss across the heat exchangers rises the speed of the pumps is increased.

In the process according to the invention the product from step c) is advantageously washed with phenol and/or recrystallised.

It is advantageous to increase the speed of the pumps over the course of the entire process according to the invention, from start-up of the heat exchangers through to removal of the deposits in the heat exchanger, by 10 to 50%. Thus, for example, the speed of the pump after one melt-off process (=start-up) is 450 rpm, and when it reaches 600 rpm a melt-off is performed in the heat exchangers.

Owing to the constant current consumption, the volume throughput circulating through the heat exchanger(s) used should fluctuate by a maximum of 15%.

In the process according to the invention the current consumption of the pump for the heat exchanger should advantageously fluctuate by a maximum of 5%.

Alternative processes exist in which the pump speed is controlled by means of the volume throughput or the pressure difference across the heat exchanger, but it is advantageous and easier to control the speed by means of the current consumption of the pump.

The invention therefore also concerns a process for producing bisphenol A in which BPA-phenol adduct crystals are crystallised out of product mixtures containing bisphenol A and phenol by means of a continuous or semi-continuous suspension crystallisation such that heat is extracted from the product mixture by pumping this product mixture through one or more heat exchangers and the product mixture spends sufficient time in a crystalliser for the supersaturation that is formed to break down, wherein the pressure difference Δp and/or the volume throughput in the heat exchanger in the cooler(s) is measured and the speed of the pump to circulate the product mixture is increased as soon as a rise in the pressure difference or a fall in the volume throughput is measured.

All standard chemical pumps known to the person skilled in the art can be used for the process according to the invention. Magnetic pumps or pumps having a mechanical shaft seal are preferred.

Tubular, plate or spiral tube heat exchangers, for example, can be used as the coolers/heat exchangers.

The adhesion of the BPA-phenol adduct to the inside of the tubes (known as fouling) increases the counter-pressure. The circulated quantity reduces as a consequence, causing the fouling to increase still further. The reduction in the circulated quantity can be detected from a reduction in the current consumption. By increasing the speed, the current consumption in the heat exchanger is kept constant or its reduction is decreased. An interconnection between current consumption and speed thus also represents an interconnection between pressure difference and speed.

Increasing the speed at the start of the reaction is not advisable because too high a speed leads to a sharp reduction in the crystal size and this makes it more difficult to filter and wash the BPA-phenol adduct crystals in a subsequent stage, resulting in a drop in quality. Thus the pump on the heat exchanger is not operated at an elevated speed right from the start of the process.

After obtaining the BPA-phenol adduct crystals, the final step of the process according to the invention is to remove the phenol from the adduct. This can be achieved in any way known to the person skilled in the art, such as distillation and/or desorption methods by heating to temperatures of >120° C., to obtain a residual content of <200 ppm phenol.

Following the separation of phenol a bisphenol A melt is obtained which can be used without prior solidification for the production of polycarbonate by the interesterification process (melt polycarbonate). However, the bisphenol A melt can also be solidified by known methods such as e.g. prilling or flaking, for sale or for further use. In addition, the melt can be dissolved in sodium hydroxide solution and used to produce polycarbonate by the interfacial polycondensation process.

In another process variant the phenol is only removed down to a residual content of ≦15% and the mixture thus obtained is sent for further processing, for example to produce polycarbonate by the melt process.

Alternatively, the BPA-phenol adduct crystals obtained from step c) can also undergo further purification before being used in step d). Purification by recrystallisation from a phenolic solution is preferred here.

After the pump speed has increased by 10% to 50% as compared with the initial state, the deposits in the heat exchanger and possibly also in the crystalliser must be removed, preferably by rapid heating to temperatures of approximately 80° C., as described in DE 100 15 014 A.

All the references described above are incorporated by reference in their entireties for all useful purposes.

While there is shown and described certain specific structures embodying the invention, it will be manifest to those skilled in the art that various modifications and rearrangements of the parts may be made without departing from the spirit and scope of the underlying inventive concept and that the same is not limited to the particular forms herein shown and described.

### EXAMPLE

### Example 1

### No Interconnection Between Speed (Power Consumption of the Pump) and Pressure Increase

A product mixture containing bisphenol A, minor components and phenol is continuously fed into a crystalliser having a heat exchanger, crystalliser cooler and circulating pump (standard chemical pump with a double mechanical shaft seal) and a partial stream of the suspension in the crystalliser is continuously circulated by the pump through the heat exchanger. A partial stream is continuously removed from the system and sent for further processing.

The following characteristics were observed:

| | Without fouling | | With fouling | |
|---|---|---|---|---|
| | (start point) | | (end point) | |
| Pump speed | 550 | rpm | 550 | rpm |
| Power consumption of pump | 60 | kW | 40 | kW |
| Circulated volume | 1500 | m³/h | 500 | m³/h |
| Δ p across heat exchanger | 1 | bar | 1.8 | bar |

The length of time between two operations to remove the deposits in the crystalliser cooler is 22 days.

TABLE 1
Influence of fouling on the circulated volume with no
interconnection between pump speed and pressure increase

| Pump speed | Δ p across heat exchanger | Circulated volume |
|---|---|---|
| 550 | 1 | 1500 |
| 550 | 1.2 | 1280 |
| 550 | 1.3 | 1100 |
| 550 | 1.45 | 900 |
| 550 | 1.6 | 650 |
| 550 | 1.8 | 500 |

### Example 2

### Interconnection Between Speed (Power Consumption of the Pump) and Pressure Increase

### As for Example 1.

| | Without fouling | | With fouling | |
|---|---|---|---|---|
| | (start point) | | (end point) | |
| Pump speed | 550 | rpm | 640 | rpm |
| Power consumption of pump | 60 | kW | 74 | kW |
| Circulated volume | 1500 | m³/h | 1300 | m³/h |
| Δ p across heat exchanger | 1 | bar | 1.8 | bar |

The length of time between two operations to remove the deposits in the crystalliser cooler is 31 days.

TABLE 2
Influence of fouling on the circulated volume with
interconnection between pump speed and pressure increase

| Pump speed | Δ p across heat exchanger | Circulated volume |
|---|---|---|
| 550 | 1 | 1500 |
| 590 | 1.2 | 1420 |
| 603 | 1.4 | 1400 |
| 612 | 1.45 | 1380 |
| 630 | 1.65 | 1340 |
| 645 | 1.8 | 1300 |

1. A process for producing bisphenol A comprising:
a) reacting phenol and acetone in the presence of a sulfonic acid ion exchanger and a sulfur-containing co-catalyst to form a product mixture comprising bisphenol A-phenol adduct;
b) subjecting said product mixture comprising bisphenol A-phenol adduct obtained in a) to continuous suspension crystallisation in one or more crystallisers, wherein said one or more crystallisers comprises at least one heat exchanger for cooling and at least one pump which pumps the suspension contained in said one or more crystallisers through said at least one heat exchanger, to obtain crystals of bisphenol A-phenol adduct;
c) separating said crystals of bisphenol A-phenol adduct obtained in b) by solid/liquid separation; and
d) removing phenol from said crystals of bisphenol A-phenol adduct obtained in c) by distillation and/or desorption to obtain bisphenol A;
wherein the current consumption of said at least one pump in b) is maintained such that the current consumption fluctuates by a maximum of ±5% and wherein the speed of said at least one pump is increased as the pressure loss across said at least one heat exchanger rises.

2. The process of claim 1, wherein the separated crystals of bisphenol A-phenol adduct obtained in c) are washed with phenol and/or recrystallised.

3. The process of claim 1, wherein said bisphenol A obtained in d) has a residual phenol content of <200 ppm.

4. The process of claim 1, wherein said bisphenol A obtained in d) has a residual phenol content of 15%.

5. The process of claim 1, wherein the speed of said at least one pump is increased by 10 to 50% from start-up of said at least one heat exchanger through to removal of the deposits in said at least one heat exchanger, relative to the initial speed.

6. The process of claim 1, wherein the volume throughput circulated through said at least one heat exchanger is maintained such that the current consumption fluctuates by a maximum of ±15%.

7. The process of claim 1, wherein the pressure difference across said at least one heat exchanger is measured and the speed of said at least one pump is increased as the pressure difference rises.
